SEQUENCE LISTING (8 sequences)

SEQ ID NO: 1 - protein, 9 aa, Artificial Sequence
  Synthetic peptide
XZXZZZXZX

SEQ ID NO: 2 - protein, 10 aa, Artificial Sequence
  Synthetic peptide
XZXZZZZXZX

SEQ ID NO: 3 - protein, 11 aa, Artificial Sequence
  Synthetic peptide
XZXZZZZZXZX

SEQ ID NO: 4 - protein, 12 aa, Artificial Sequence
  Synthetic peptide
XZXZZZZZZXZX

SEQ ID NO: 5 - protein, 13 aa, Artificial Sequence
  Synthetic peptide
XZXZZZZZZZXZX

SEQ ID NO: 6 - protein, 11 aa, Artificial Sequence
  Synthetic peptide
KLKLLLLLKLK

SEQ ID NO: 7 - protein, 26 aa, Artificial Sequence
  Synthetic peptide
ICICICICICICICICICICICICIC

SEQ ID NO: 8 - DNA, 24 bp, Artificial Sequence
  Synthetic primer
tcgtcgttttgtcgttttgtcgtt

(19) United States
(12) Patent Application Publication
(10) Pub. No.: US 2011/0236414 A1
(43) Pub. Date: Sep. 29, 2011

(54) Bacterial Polysaccharide-Polypeptide Conjugate Compositions

(75) Inventors: Thorunn Olafsdottir, Reykjavik (IS); Ingileif Jonsdottir, Reykjavik (IS); Eszter Nagy, Vienna (AT); Alena Egyed, Vienna (AT); Karen Lingnau, Vienna (AT)

(73) Assignee: INTERCELL AG, Vienna (AT)

(21) Appl. No.: 13/058,161

(22) PCT Filed: Aug. 7, 2009
(86) PCT No.: PCT/EP09/60271

§ 371 Date: Feb. 8, 2011

(30) Foreign Application Priority Data
Aug. 8, 2008 (EP) 08162061.9

(51) Int. Cl. A61K 39/385; A61K 39/39; A61P 37/04; A61P 31/04

(52) U.S. Cl. 424/197.11; 424/278.1

(57) ABSTRACT

The present invention relates to improved bacterial polysaccharide-polypeptide conjugate compositions, pharmaceutical compositions comprising such bacterial polysaccharide-polypeptide conjugate compositions, and the use of such compositions. Furthermore, the invention relates to the use of a combination of a peptide of the formula R₁—XZXZ_{N}XZX—R₂ and an immunostimulatory deoxynucleic acid containing deoxyinosine and/or deoxyuridine residues. The present invention also provides methods for the prevention of a bacterial infection, especially an infection with Streptococcus pneumoniae (S. pneumoniae).

The present invention relates to improved bacterial polysaccharide-polypeptide conjugate compositions, pharmaceutical compositions comprising such bacterial polysaccharide-polypeptide conjugate compositions, and the use of such compositions. Furthermore, the invention relates to the use of a combination of a peptide of the formula R₁—XZXZ_{N}XZX—R₂ and an immunostimulatory deoxynucleic acid containing deoxyinosine and/or deoxyuridine residues. The present invention also provides methods for the prevention of a bacterial infection, especially an infection with Streptococcus pneumoniae (S. pneumoniae).

Bacteria, typically a few micrometres in length, have a wide-range of shapes, ranging from spheres to rods to spirals. They are ubiquitous in every habitat on Earth. There are approximately ten times as many bacterial cells as human cells in the human body, with large numbers of bacteria on the skin and in the digestive tract. Although the vast majority of these bacteria are rendered harmless or beneficial by the protective effects of the immune system, a few pathogenic bacteria cause infectious diseases, including cholera, syphilis, anthrax, leprosy and bubonic plague. The most common fatal bacterial diseases are respiratory infections, with tuberculosis alone killing about 2 million people a year. In developed countries, antibiotics are used to treat bacterial infections and in various agricultural processes, so antibiotic resistance is becoming common.

By combining morphology and Gram-staining, most bacteria can be classified as belonging to one of four groups (Gram-positive cocci: Staphylococci and Streptococci; Gram-positive bacilli: Corynebacteria, Bacillus anthracis, Listeria monocytogenes; Gram-negative cocci: Neisseriae; and Gram-negative bacilli: Salmonella, Shigella, Campylobacter, Vibrio, Yersinia, Pasteurella, Pseudomonas, Brucella, Haemophilus, Legionella, Bordetella). Some organisms are best identified by stains other than the Gram stain, particularly Mycobacteria or Nocardia, which show acid-fastness on Ziehl-Neelsen or similar stains. Other organisms may need to be identified by their growth in special media, or by other techniques, such as serology.

Polysaccharide encapsulated bacteria, also referred to as encapsulated bacteria, are a group of bacteria that have an outer covering, a capsule, made of polysaccharides. It includes such human pathogens as Haemophilus influenzae type b (Hib), Neisseria meningitidis, Streptococcus pneumoniae and Group B Streptococci (GBS).

The first three pathogens are the most important causes of childhood invasive bacterial diseases, including meningitis. The high susceptibility to encapsulated bacteria in early childhood is caused by the inability of infants and young children to mount antibodies to the capsular polysaccharides. Polysaccharides are traditionally viewed as T-independent antigens with a number of unique and important immunological properties that are not encountered when inducing an immune response to proteins. These properties include no overt requirement for the presence of T cells to induce an immune response, dominance of IgM with low IgG, in particular low IgG2 response, failure to induce immunological memory following immunization, an absence of affinity maturation following immunization, and poor immunogenicity in infants, the elderly and the immunocompromised. These properties of carbohydrates have precluded the use of pure carbohydrate vaccines in those patients most at risk. Conjugate vaccine technology, where a carbohydrate antigen is covalently coupled chemically to a protein carrier, has overcome the limitations of carbohydrates as vaccine antigens by rendering the carbohydrate moiety of such vaccines T-cell dependent and immunogenic, even in the very young (for review see e.g. Finn, British Medical Bulletin, 2004, 70:1-14).

Hib vaccine was the first conjugate vaccine developed for the prevention of invasive disease caused by Haemophilus influenzae type b bacteria. Due to routine use of the Hib vaccine in the U.S. from 1980 to 1990, the incidence of invasive Hib disease has decreased from 40-100 per 100,000 children down to 1.3 per 100,000. The dramatic success of the Hib vaccines has demonstrated the potential value of conjugate vaccines. Similar technology has been applied to a number of other vaccines, including N. meningitidis (groups A and C) and S. pneumoniae vaccines.

Group B Streptococcus (GBS) remains a major cause of sepsis and pneumonia in neonates. GBS vaccines are in the early stages of clinical development as prenatal or antenatal vaccines.

Streptococcus pneumoniae, or pneumococcus, is a significant human pathogen which can cause a wide variety of disease in infants, children, healthy adults, the immunocompromised, and the elderly, including meningitis, pneumonia, bacteremia, sinusitis, and others. In the United States alone, over 800,000 cases of pneumonia annually require hospitalization. Treatment usually consists of penicillin-class or third-generation cephalosporin antibiotics; in the United States, penicillin is the standard treatment. However, antibiotic resistance is a growing concern, and resistance to penicillin and macrolides continues to increase. Resistance has been additionally seen to fluoroquinolones and amoxicillin, and penicillin resistance has been linked to an increase in patient mortality. The list of antibiotics that can be used to combat pneumococcal infection, particularly nosocomial infection, is growing shorter.

Annually up to 1 million children die of pneumococcal diseases worldwide, children under 2 years of age in the developing countries being the largest group according to WHO.

The marketed 23-valent polysaccharide vaccine Pneumovax II is only appropriate for adults and generally should be a single lifetime dose (high risk side effects if repeated). Children under the age of two years fail to mount an adequate response to the 23-valent vaccine, and instead a Pneumococcal Conjugated Vaccine (PCV) must be used.

A seven-valent PCV (Prevnar/Prevenar) comprises seven PPSs conjugated to CRM197, a non-toxic variant of diphtheria toxin, and aluminum phosphate adjuvant. The vaccine was licensed in the year 2000 and has proven efficacious in reducing invasive pneumococcal disease and pneumonia, as well as otitis media. Herd immunity is also generated as nasopharyngeal colonization and thus spreading is reduced. PCVs with up to at least 13 serotypes are being developed. Conjugation of pneumococcal polysaccharide to a carrier polypeptide increases its immunogenicity by converting it from a T-cell independent (TI) antigen of type 2 (TI-2) to become a T-cell dependent (TD) antigen. TD antigens have the advantage over TI antigens that immune response can occur at or shortly after birth, they induce affinity maturation of antibodies and immunological memory is generated. As a result, PCV-7 needs to be administrated several times before it yields protective antibody (Ab) levels in infants and young children (see Cutts et al., Lancet, 2005, 365(9465):1139-46; Black et al., Pediatr Infect Dis J, 2002, 21(9):810-5; Black et al., Pediatr Infect Dis J, 2007, 26(9):771-7). This is a shortcoming especially in developing countries, where visits to health-care centres are scarce.

Therefore, it is important to develop safe and effective early life vaccination strategies based on as few administrations as possible.

Type 1 immune responses are limited in the neonatal period and early infancy but increase with age. Moreover, since bacterial vaccines, and especially the pneumococcal vaccines, which are currently in clinical development or on the market are predominantly eliciting type 2 responses in infants and children, also a need exists to provide improved vaccines which show a type 1 directed immune response or vaccines which allow—in addition to a type 2 response—also a significant type 1 immune reaction. Thus, pneumococcal vaccines already available should be provided in an improved form which allows the induction of a profound type 1 response and enhanced type 2 response.

Thus, the object of the present invention is to provide improved bacterial polysaccharide-polypeptide conjugate compositions.

The present invention provides improved compositions against bacterial infections, comprising at least one polysaccharide-peptide conjugate antigen, at least one peptide of the formula R₁—XZXZ_{N}XZX—R₂ and st least one immunostimulatory deoxynucleic acid molecule containing deoxyinosine and/or deoxyuridine residues.

In one aspect, the present invention therefore provides a composition comprising
at least one polysaccharide-polypeptide conjugate,
at least one peptide comprising a sequence R₁—XZXZ_{N}XZX—R₂, whereby N is a whole number between 3 and 7, preferably 5, X is a positively charged natural and/or non-natural amino acid residue, Z is an amino acid residue selected from the group consisting of L, V, I, F and/or W, and R₁ and R₂ are selected independently one from the other from the group consisting of —H, —NH₂, —COCH₃, —COH, a peptide with up to 20 amino acid residues or a peptide reactive group or a peptide linker with or without a peptide; X—R₂ may be an amide, ester or thioester of the C-terminal amino acid residue of the peptide (in the following also referred to as “Peptide A”), and
at least one immunostimulatory oligodeoxynucleic acid molecule (ODN) having the structure according to the formula (I)

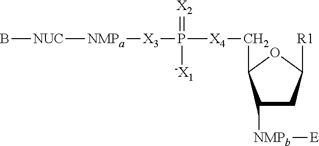

wherein
R1 is selected from hypoxanthine and uracile,

### any X is O or S,

any NMP is a 2′ deoxynucleoside monophosphate or monothiophosphate, selected from the group consisting of deoxyadenosine-, deoxyguanosine-, deoxyinosine-, deoxycytosine-, deoxyuridine-, deoxythymidine-, 2-methyl-deoxyinosine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine-monophosphate or -monothiophosphate, NUC is a 2′ deoxynucleoside, selected from the group consisting of deoxyadenosine-, deoxyguanosine-, deoxyinosine-, deoxycytosine-, deoxyinosine-, deoxythymidine-, 2-methyl-deoxyuridine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine, a and b are integers from 0 to 100 with the proviso that a+b is between 4 and 150, and B and E are common groups for 5′ or 3′ ends of nucleic acid molecules (in the following also referred to as “I-/U-ODN”).

The polysaccharide-polypeptide conjugate according to the present invention comprises at least one polysaccharide and at least one polypeptide.

The polysaccharide according to the invention is preferably a bacterial capsular polysaccharide. Capsular polysaccharides can be prepared by standard techniques known to those of skill in the art.

In an embodiment, the polysaccharide is a S. pneumoniae capsular polysaccharide. In another embodiment, the S. pneumoniae capsular polysaccharide is selected from the group consisting of serotype 1, 2, 3, 4, 5, 6B, 7F, 8, 9N, 9V, 10A, 11A, 12F, 14, 15B, 17F, 18C, 19A, 19F, 20, 22F, 23F and 33F, representing the 23 pneumococcal serotypes causing the vast majority of pneumococcal disease in all age groups, out of the more than 90 serotypes known so far.

The term “polysaccharide” as used herein refers to polysaccharides and/or oligosaccharides. Polysaccharides are isolated from bacteria and may be depolymerized to a preferred size range by known methods (see for example EP 497524 and EP 497525). Oligosaccharides have a low number of repeat units (typically 5-30 repeat units) and are typically hydrolysed polysaccharides.

Capsular polysaccharides of Streptococcus pneumoniae comprise repeating oligosaccharide units which may contain up to 8 sugar residues. For a review of the oligosaccharide units for the key Streptococcus pneumoniae serotypes see Jones et al., An. Acad. Bras. Cienc, 2005, 77(2):293-324. In one embodiment, a capsular saccharide antigen may be a full length polysaccharide, however in others it may be one oligosaccharide unit, or a shorter than native length saccharide chain of repeating oligosaccharide units.

Full length polysaccharides may be “sized” or “depolymerized”, i.e. their size may be reduced by various methods known in the art (as described above). The term “depolymerization” includes partial depolymerization.

The depolymerization of the polysaccharides may then be followed by an activation step prior to conjugation to a carrier polypeptide. By “activation” is meant chemical treatment of the polysaccharide to provide chemical groups capable of reacting with the carrier polypeptide. Appropriate methods are known in the art.

By “polypeptide” or “protein” is meant any chain of amino acids, regardless of the size or post-translational modification. Suitable polypeptide carriers include, but are not limited to, diphtheria toxin, diphtheria toxoid, CRM197, tetanus toxoid, pertussis toxoid, E. coli LT, E. coli ST, exotoxin A, outer membrane complex c (OMPC), porin, transferrin binding protein, pneumolysis, pneumococcal surface protein A (PspA), pneumococcal adhesin protein (PsaA), ovalbumin, keyhole limpit hemocyanin (KLH), bovine serum albumin (BSA) or purified protein derivative of tuberculin (PPD), and the like. Carrier polypeptides are preferably polypeptides that are non-toxic and non-reactogenic and obtainable in sufficient amount and purity. In a particularly preferred embodiment, the carrier polypeptide comprises a tetanus toxoid. In another preferred embodiment, the carrier polypeptide comprises a derivative of any of the above mentioned carrier polypeptides, for example a subunit or a mutated version of E. coli LT, such as LT or the A subunit of LT (LTA) having an amino acid substitution at the position of aa 192 (e.g. LTG 192, LTT 192, LTS 192, LTA 192), LTK 63 LTR 72, or other mutants as described e.g. in WO 98/42375, WO 02/64162, U.S. Pat. No. 4,761,372, U.S. Pat. No. 5,308,835.

The polypeptides may also contain elongations either at the carboxy- or at the amino-terminus of the polypeptide facilitating interaction with the polycationic compound(s) or the immunostimulatory compound(s).

Furthermore, the polypeptides may also be derivatized to include molecules enhancing antigen presentation and targeting of antigens to antigen presenting cells.

The polypeptide may be activated prior to conjugation.

The nature and size of the saccharide, the nature and size of protein or polypeptide, the ratio of the saccharide and protein/polypeptide, as well as other factors and conditions for the preparation of a conjugate according to the present invention can be determined by a skilled person, as described e.g. in Robbins et al., JAMA, 1996, 276(14):1181-5. For example, for a pneumococcus polysaccharide and a tetanus toxoid a preferred ratio is approximately 2:1.

By “conjugate” is meant a compound in which the polysaccharide is covalently linked to a carrier polypeptide. There are many conjugation reactions known in the prior art that have been employed for covalently linking polysaccharides to polypeptides in order to produce a polysaccharide-polypeptide conjugate. Three of the more commonly employed methods include: 1) reductive amination, wherein the aldehyde or ketone group on one component of the reaction reacts with the amino or hydrazide group on the other component, and the C—N double bond formed is subsequently reduced to C—N single bond by a reducing agent; 2) cyanylation conjugation, wherein the polysaccharide is activated either by cyanogens bromide (CNBr) or by 1-cyano-4-dimethylammoniumpyridinium tetrafluoroborate (CDAP) to introduce a cyanate group to the hydroxyl group, which forms a covalent bond to the amino or hydrazide group upon addition of the protein component; and 3) a carbodiimide reaction, wherein carbodiimide activates the carboxyl group on one component of the conjugation reaction, and the activated carbonyl group reacts with the amino or hydrazide group on the other component. These reactions are also frequently employed to activate the components of the conjugate prior to the conjugation reaction.

The polysaccharide may be conjugated to the polypeptide directly or via a linker. Linkage via a linker group may be made using any known procedure, for example, the procedures described in U.S. Pat. No. 4,882,317 and U.S. Pat. No. 4,695,624. Suitable linkers include carbonyl, adipic acid, B-propionamido (WO 00/10599), nitrophenyl-ethylamine (Geyer et al., Med. Microbiol. Immunol, 1979, 165:171-288), haloacyl halides (U.S. Pat. No. 4,057,685), glycosidic linkages (U.S. Pat. No. 4,673,574; U.S. Pat. No. 4,761,283; U.S. Pat. No. 4,808,700), 6-aminocaproic acid (U.S. Pat. No. 4,459,286), ADH (U.S. Pat. No. 4,965,338), C₄ to C₁₂ moieties (U.S. Pat. No. 4,663,160), etc.

After conjugation of the polysaccharide to the carrier polypeptide, the polysaccharide-polypeptide conjugate may be purified (enriched with respect to the amount of polysaccharide-polypeptide conjugate) by a variety of techniques known in the art. One goal of the purification step is to remove the unbound polysaccharide and/or polypeptide from the polysaccharide-polypeptide conjugate. Methods for purification include e.g. ultrafiltration in the presence of ammonium sulfate, size exclusion chromatography, density gradient centrifugation, and hydrophobic interaction chromatography.

In an embodiment of the present invention, the composition may comprise two or more polysaccharide-polypeptide conjugates. In another embodiment, the composition comprises two or more polysaccharide-polypeptide conjugates, wherein the polysaccharide moieties are derived from different serotypes of the same bacteria, especially of different S. pneumoniae serotypes. In still another embodiment, the composition comprises at least one polysaccharide-polypeptide conjugate having a polysaccharide moiety from one bacteria, especially S. pneumoniae, and another polysaccharide-polypeptide conjugate having a polysaccharide moiety from a different pathogen. For example, at least one S. pneumoniae polysaccharide-polypeptide conjugate can be combined with at least one polysaccharide-polypeptide conjugate derived from N. meningitidis types A, C, W, Y; H. influenzae type B; S. aureus; S. epidermidis; Group B Streptococcus; Group A Streptococcus; Bordetella pertussis, Clostridium tetani, Corynebacterium diphtheriae, Salmonella typhi, etc. Preferably it is N. meningitidis (types A and/or C are most preferred), and/or H. influenzae type B. Methods for combining several polysaccharide-polypeptide conjugates to multivalent compositions are well known in the art and are described e.g. in WO 03/51392.

The composition and/or pharmaceutical composition according to the present invention may further contain additional adjuvants, especially an Al(OH)₃ adjuvant (Alum).

Alum, as meant herein includes all forms of Al³⁺ based adjuvants used in human and animal medicine and research. Especially, it includes all forms of aluminum hydroxide as defined in e.g. the chemical encyclopedia Römpp, 10th Ed., pages 139/140, gel forms thereof, aluminum phosphate, etc.

This is especially preferred for pharmaceutical compositions which are in clinical development or are already on the market and which contain such Al(OH)₃ adjuvants. In such a case, the combination of at least one Peptide A and at least one I-/U-ODN according to the present invention may simply be added to such an existing pharmaceutical composition.

In an embodiment, Peptide A is a polycationic peptide. The polycationic peptide to be used according to the present invention may be any polycationic compound which shows the characteristic effect according to the WO 97/30721. Preferred polycationic compounds are selected from basic polypeptides, organic polycations, basic polyaminoacids or mixtures thereof. These polyaminoacids should have a chain length of at least 4 amino acid residues. Especially preferred are substances containing peptidic bounds, like polylysine, polyarginine and polypeptides containing more than 20%, especially more than 50% of basic amino acids in a range of more than 8, especially more than 20, amino acid residues or mixtures thereof. Preferably these polypeptides contain between 20 and 500 amino acid residues, especially between 30 and 200 residues. The polycationic peptide according to the invention may also be a cationic antimicrobial peptide and may be of prokaryotic or eukaryotic origin (see e.g. WO 02/13857). Such cationic antimicrobial peptides may also belong to the class of naturally occurring antimicrobial peptides. Such host defense peptides or defensives are also a preferred form of the polycationic polymer according to the present invention. Generally, a compound allowing as an end product activation (or down-regulation) of the adaptive immune system, preferably mediated by APCs (including dendritic cells) is used as polycationic polymer. Other preferred polycations and their pharmaceutical compositions are described in WO 97/30721, WO 99/38528, WO 01/93903, and WO 02/32451. In an especially preferred embodiment of the invention, the polycationic peptide comprises at least two LysLeuLys motifs. In another embodiment, the polycationic peptide is KLKLLLLLKLK.

These polycationic compounds may be produced chemically or recombinantly or may be derived from natural sources.

Furthermore, also neuroactive compounds, such as (human) growth hormone (as described e.g. in WO 01/24822) may be used as immunostimulants (adjuvants).

Polycationic compounds derived from natural sources include HIV-REV or HIV-TAT (derived cationic peptides, antennapedia peptides, chitosan or other derivatives of chitin) or other peptides derived from these peptides or proteins by biochemical or recombinant production. Other preferred polycationic compounds are cathelin or related or derived substances from cathelicidin, especially mouse, bovine or especially human cathelicidins and/or cathelicidins. Related or derived cathelicidin substances contain the whole or parts of the cathelicidin sequence with at least 15-20 amino acid residues. Derivations may include the substitution or modification of the natural amino acids by amino acids which are not among the 20 standard amino acids. Moreover, further cationic residues may be introduced into such cathelicidin molecules. These cathelicidin molecules are preferred to be combined with the antigen/vaccine composition according to the present invention. However, these cathelin molecules surprisingly have turned out to be also effective as an adjuvant for a antigen without the addition of further adjuvants. It is therefore possible to use such cathelicidin molecules as efficient adjuvants in vaccine formulations with or without further immunactivating substances.

In an embodiment, the at least one immunostimulatory oligodeoxynucleic acid molecule is Oligo(dIdC)₁₃. Further deoxynucleotides are described e.g. in WO 01/93903, WO 01/93905, and WO 02/95027.

The term “Oligo(dIdC)₁₃” as used in the present invention means a phosphodiester backboned single-stranded DNA molecule containing 13 deoxy (inosine-cytosine) motifs, also defined by the term [oligo-d(IC)₁₃]. The exact sequence is 5′-dIdCdIdCdIdCdIdCdIdCdIdCdIdCdIdCdIdCdIdCdIdCdIdCdIdC-3′. Oligo(dIdC)₁₃ can also be defined by the terms (oligo-dIC₂₆); oligo-dIC₂₆₋ₘₑᵣ; oligo-deoxy IC, 26-mer; or oligo-dIC, 26-mer, as specified for example in WO 01/93903 and WO 01/93905.

It is also within the present invention that any of the aforementioned polycationic compounds is combined with any of the immunostimulatory oligodeoxynucleic acid molecules as aforementioned.

The composition according to the present invention preferably contains as Peptide A KLKLLLLLKLK, and as I-/U-ODN oligo d(IC)₁₃. The combination of Peptide A and oligo d(IC)₁₃ is also referred to as IC31®.

The composition according to the present invention may further (or even instead of the I-/U-ODN) contain an oligodeoxynucleotide containing a CpG-motif as immunomodulating nucleic acids. The immunomodulating nucleic acids to be used according to the present invention can be of synthetic, prokaryotic and eukaryotic origin. In the case of eukaryotic origin, DNA should be derived from, based on the phylogenetic tree, less developed species (e.g. insects, but also others). In a preferred embodiment of the invention the immunogenic oligodeoxynucleotide (ODN) is a synthetically produced DNA-molecule or mixtures of such molecules. Derivates or modifications of ODNs such as thiophosphate substituted analogues (thiophosphate residues substitute for phosphate) as for example described in U.S. Pat. No. 5,723,335 and U.S. Pat. No. 5,663,153, and other derivatives and modifications, which preferably stabilize the immunostimulatory composition(s) but do not change their immunological properties, are also included. A preferred sequence motif is a six base DNA motif containing an (unmethylated) CpG dinucleotide flanked by two 5′ purines (R) and two 3′ pyrimidines (Y), which can be depicted by the following general formula: 5′-R-R-C-G-Y-Y-3′. The CpG motifs contained in the ODNs according to the present invention are more common in microbial than higher vertebrate DNA and display differences in the pattern of methylation.

Preferred palindromic or non-palindromic ODNs to be used according to the present invention are disclosed e.g. in WO 01/93905, WO 02/95027, EP 0468520, WO 96/02555, WO 98/16247, WO 98/18810, WO 98/37919, WO 98/40100, WO 98/52581, WO 98/52962, WO 99/51259 and WO 99/56755 all incorporated herein by reference. ODNs/DNAs may be produced chemically or recombinantly or may be derived from natural sources. Preferred natural sources are insects.

The composition according to the present invention may preferably contain a polycationic peptide and an I-/U-ODN or an oligodeoxynucleotide containing a CpG-motif in combination. Of course, also mixtures of different immunostimulatory nucleic acids (I-/U-ODNs, CpG-ODNs, etc.) and Peptide A variants may be used according to the present invention, optionally together with one or more further adjuvants.

The polysaccharide-peptide conjugate(s) may be mixed with the adjuvant(s) according to the present invention or otherwise specifically formulated e.g. as liposome retard formulation, etc.

With the present invention it is also possible to significantly improve bacterial vaccines, especially S. pneumoniae vaccines, being already in clinical development or on the market simply by additionally providing the combination of the two types of adjuvants according to the present invention.

In another aspect, the present invention relates to a pharmaceutical composition comprising the composition according to the invention, and optionally a pharmaceutically acceptable carrier or excipient. The pharmaceutical composition can be used for the prevention of a bacterial infection, especially an infection with S. pneumoniae. In an especially preferred embodiment, the pharmaceutical composition according to the invention is for administration to a human toddler, infant, or neonate. In another preferred embodiment, the pharmaceutical composition according to the invention is for administration to a human subject with at least 60, especially preferred at least 65 years of age. In still another embodiment, the pharmaceutical composition according to the invention is for administration to an adult immunocompromized human subject.

Another aspect of the invention relates to the use of a composition according to invention for the manufacture of a pharmaceutical composition for the prevention of a bacterial infection, especially an infection with S. pneumoniae. In an embodiment, the composition is used for the manufacture of a pharmaceutical composition for the prevention of an infection in a human toddler, infant, or neonate. In another preferred embodiment, the composition is used for the manufacture of a pharmaceutical composition for the prevention of an infection in a human subject with at least 60, especially preferred at least 65 years of age. In still another embodiment, the composition is used for the manufacture of a pharmaceutical composition for the prevention of an infection in an adult immunocompromized human subject.

In still another aspect, the invention relates to the use of a combination of Peptide A and a I-/U-ODN according to the invention to improve the protective efficacy of a composition comprising a polysaccharide-peptide conjugate.

In another aspect, the present invention relates to the use of a combination of Peptide A and a I-/U-ODN according to the invention to improve the antigen-specific type 1 response of a composition comprising a polysaccharide-polypeptide conjugate, and at the same time preserving or enhancing the type 2 response of said composition. Specifically, the antigen-specific type 1 response of a vaccine against a bacterial pathogen, especially S. pneumoniae, can be improved and at the same time the type 2 response, of said vaccine can be preserved.

Another aspect relates to a method for the prevention of a bacterial infection, especially an infection with S. pneumoniae, in a subject, comprising the step of administering a prophylactically effective amount of a composition according to the invention or a pharmaceutical composition according to the invention to the subject in need thereof.

The amount of the composition and/or pharmaceutical composition of the invention to be administered to a human or animal and the regime of administration can be determined in accordance with standard techniques well known to those of ordinary skill in the pharmaceutical and veterinary arts taking into consideration such factors as the particular antigen, the adjuvant(s), the age, sex, weight, species and condition of the particular animal or patient, and the route of administration.

In one embodiment, the amount of polysaccharide-polypeptide carrier to provide an efficacious dose for vaccination against a bacterial infection, especially against an S. pneumoniae infection, can be from 0.02 μg to 5 μg per kg body weight. In a preferred composition and method of the present invention the dosage is from 0.1 μg to 3 μg per kg of body weight. For pneumococcal conjugates, a preferred polysaccharide-polypeptide conjugate dosage may range between 2 μg (for most serotypes) to 4 μg (poorly immunogenic serotypes) per dose for human infants. For meningococcal C conjugates higher dosages are preferred, such as e.g. 10 μg/dose.

In an embodiment, the composition is administered 2-4 times preferably in infancy. In another embodiment, the composition is administered at 6-8 week intervals. In still another embodiment, the composition is administered to a human subject at the age of 2, 4 (or 3.5), and 6 months; optionally with a booster dose within the second year of life. In yet another embodiment, the composition may be administered to a human subject at the age of 6, 10, and 14 weeks (e.g. in developing countries).

In a preferred embodiment, the composition is administered only once.

The composition may be administered subcutaneously, intramuscularly, intravenously, parenterally, topically, intradermally, or transdermally.

Prevention in the context of the present invention refers to prophylactic or preventative measures, wherein the object is to prevent or slow down (lessen) the transmission of the targeted pathologic condition or disorder. Subjects in need of prevention include those prone to have the disorder or those in whom the disorder is to be prevented. The prevention can be direct (protecting by eliciting protective immune response in the vaccinated individual) or indirect by reducing transmission. For instance, for PCV reduced nasopharyngeal carriage has been demonstrated and a strong heard effect has been shown in all age groups in the US following introduction of the routine infant vaccination.

The terms “vaccinated” or “vaccination” as used herein comprises the administration of an antigenic pharmaceutical composition to a subject to induce a protective immune response.

The subject as described herein may be a human or any animal. In a preferred embodiment, the subject is a human. In another preferred embodiment, the human subject is a toddler, e.g. a human up to 2 or 2.5 years of age, an infant, e.g. a human within the first year of life, more preferably a neonate, e.g. a human within the first month of life.

In another embodiment, the human subject is at least 60, especially preferred at least 65 years of age. In still another embodiment, the human subject is an adult immunocompromized subject.

### FIGURE LEGENDS

FIG. 1: PPS1 specific Ab responses following one immunization with Pnc1-TT alone or in combination with IC31® LD, IC31® HD or CpG2006. Panel A shows statistical comparison between the immunized groups 4 weeks after the immunization. Unimmunized group was used as a negative control. Groups marked with * have significantly higher Ab levels than the unimmunized group. FIG. 1B shows the kinetic of the Ab response 2, 3 and 4 weeks after priming. The results are shown for one of two comparable experiments (N=8/group).

FIG. 2: Colony forming units (CFU) in blood (A) and lungs (B) 24 h after challenge with S. pneumoniae serotype 1. CFU/mL for each mouse (N=8/group) is shown and p values where differences between the groups were statistically significant. Groups marked with * have significantly reduced CFU/mL compared to the saline group. The results are shown for one of two experiments with comparable results.

FIG. 3: Correlation between PPS1 specific antibody levels (EU/mL) and pneumococcal CFU/mL blood and lungs 24 h after i.n. challenge with serotype 1 pneumococci. Each symbol represents one mouse. The results are shown for one of two comparable experiments.

FIG. 4: PPS1 specific Ab responses following two immunizations with Pnc1-TT alone or in combination with LD or HD IC31®. FIG. 4A shows statistical comparison between the immunized groups 2 weeks after the 2nd immunization. Unimmunized group was used as a negative control. Groups marked with * have statistically higher Ab levels than the unimmunized group. FIG. 4B shows the kinetic of the Ab response 2, 3 and 4 weeks after priming and the arrow indicates the time of the 2nd immunization. The results are shown for one representative of three comparable experiments (N=8/group).

### EXAMPLES

### Materials and Methods

### Mice

Adult NMRI mice were purchased from M&B AS (Ry, Denmark) and allowed to adapt for one week before matching. They were kept in micro-isolator cages with free access to commercial food pellets and water, and housed under standardized conditions at the institute of Experimental Pathology at Keldur (Reykjavik, Iceland) with regulated daylight humidity and temperature. Breeding cages were checked daily for new births, and the pups were kept with their mother until weaning at the age of 4 weeks.

### Vaccine and Adjuvants

Pneumococcal polysaccharide (PP) of serotype 1 was conjugated with tetanus toxoid (Pnc1-TT) by the Centre d'Immunology Pierre Farbe (St. Julien en Genevois France). IC31® was produced by Intercell AG (Vienna, Austria) as previously described (Schellack et al., Vaccine, 2006, 24(26): p. 5461-72). CpG-ODN (CpG2006, TCGTCGTTTTGTCGTTTTGTCGTT) was purchased from Oligos Etc., Inc. (Willsonville, Oreg.; USA). Pnc1-TT and adjuvants were mixed 1 hour prior to immunization.

### Immunization

Neonatal (7 days old) mice (8 per group) were injected subcutaneously (s.c.) in the scapular girdle region with 0.5 μg of Pnc1-TT with or without IC31® low dose (15.75 nmol KLK and 0.63 nmol ODN1a), IC31® high dose (90 nmol KLK and 3.6 nmol ODN1a) or 20 μg CpG2006. Saline was added to the formulations so that each mouse received 50 μl of solution. Second dose of 100 μl of solution was given 16 days after priming. Two identical experiments were performed for one immunization and three experiments for two immunizations.

### Blood Samples

Three weeks after the priming and then weekly throughout the experiment, mice were bled from the tail-vein, serum isolated and stored at −20° C. until use.

### Pneumococci and Mouse Challenge

Two weeks after the second immunization or four weeks after priming mice were challenged intranasally with S. pneumoniae serotype 1. Stock solution of S. pneumoniae serotype 1 (ATCC 6301) was maintained in tryptose broth +20% glycerol at −70° C. One day before challenge, the bacteria was plated on blood agar made of Tryptonse Soya Agar (Oxoid, Cambridge, UK) supplemented with Gentamicin and horse serum (Keldur, Reykjavik, Iceland) and incubated at 37° C. in 5% CO₂ over night. Next morning isolated colonies were transferred to Todd Hewitt broth (Oxoid), cultured at 37° C. to a log-phase for 3.5 h and resuspended in sterile saline. Serial 10-fold dilutions were plated on blood agar to determine the challenge dose, which was ˜3−4×10⁷ colony forming units (CFU) in 50 μl.

Twenty four hours after intranasal challenge the mice were sacrificed, blood samples taken from the tail vein and ten-fold serial dilutions plated on blood agar that included Staph/Strep selective supplement containing nalidixic acid and solistin sulphate (Oxoid) which was incubated at 37° C. in 5% CO₂ over night. Bacteremia was determined as the number of CFU per ml of blood. Lungs were removed, homogenized and diluted to 3 mL PBS and serial dilutions plated on blood agar, which was incubated for 48 h at 37° C. under anaerobic conditions. Pneumococcal lung infection was expressed as CFU per ml of lung homogenate. Depending on the first dilution used, the detection limit was 2.2 CFU/ml lung homogenate and 1.3 CFU/ml blood.

### Antibodies to PPS

PPS specific antibodies (IgG and IgG subclasses) were measured by ELISA (Jakobsen et al., Vaccine, 2001, 19(25): p. 3331-46). Briefly microtiter plates (MaxiSorp; Nunc) were coated with 100 μl/well of 10 μg/ml PPS1 (American Tissue Culture Collection, ATCC, Rockville, Md.) PBS for 5 h at 37° C. and kept at 4° C. until use. Serum samples were diluted 1:50 in PBS-Tween and incubated in 500 μg/ml of Cell Wall Polysaccharide (CWPS) (Statens Serum Institute, Copenhagen, Denmark) for 30 minutes to neutralize antibodies to CWPS. Then plates were washed 3 times with PBS-Tween and the neutralized serum samples added, serially diluted and incubated in duplicates for 2 hours at RT, washed as before and incubated for 2 h at RT with horseradish peroxidase conjugated goat anti-mouse IgG, IgG1, IgG2a, IgG2b and IgG3 (Southern Biotechnology Associates Inc., Birmingham, Al.) antibodies diluted 1:5000 in PBS-Tween. The plates were washed as before and the enzyme reaction developed by 3,3′,5,5′-tetramethylbenzidine peroxidase substrate (Kirkegaard & Perry Laboratories, Gaithersburg, Md.). After 10-30 minutes the reaction was stopped by adding 100 μA of 0.18 M H₂SO₄ to each well. The absorbance was measured at 450 nm in an ELISA spectrophotometer (Titertek Multiscan Plus MK II). Serial dilutions of a reference serum, obtained by hyper-immunizing adult mice with the conjugate vaccine, was included on each microtiter plate. The titer of the reference serum corresponded to the inverse of the serum dilution giving an optical density of 1.0. The titers of the test serum samples were calculated from the reference serum and based on a minimum of four data points and parallelism between the serum samples and the reference curve. The detection limit was 1.0 EU/mL. The results are expressed as mean of log ELISA units per ml (EU/ml)±SD.

### Statistical Analysis

Non-parametric Mann-Whitney Rank Sum test was used for statistical analysis using the program Sigma Stat. A P value of <0.05 was considered statistically significant.

### Example 1

### One Dose of IC31 Enhanced Immunogenicity of Pnc1-TT and Protective Efficacy in Neonatal Mice

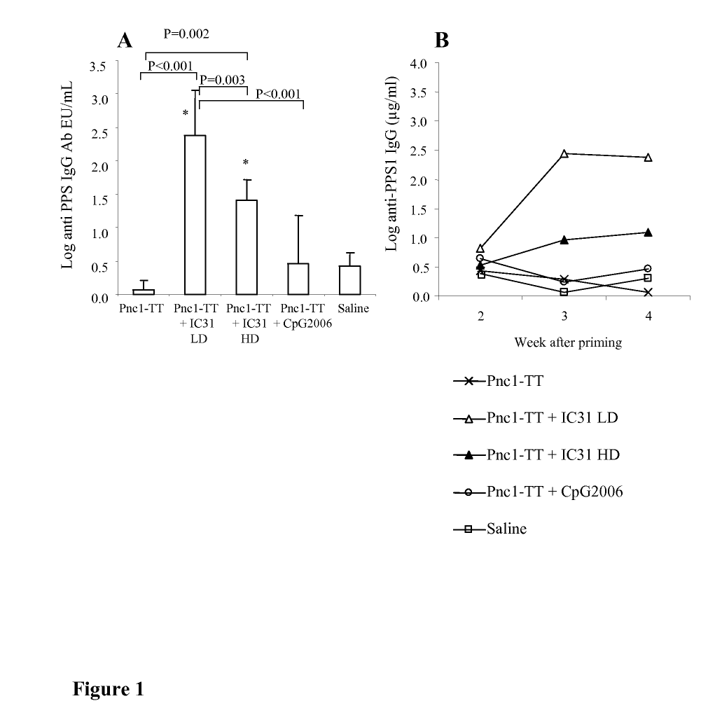

Neonatal mice (7 days old; 8 mice per group) were immunized once with Pnc1-TT with or without IC31® low dose (LD), IC31® high dose (HD) or CpG2006. One group received saline as a control. For practical reasons the mice were not bled until three weeks of age or two weeks after priming, and then weekly thereafter. The combinations of Pnc1-TT with either LD or HD of IC31® elicited significantly higher PPS1-specific Ab response than Pnc1-TT alone (P<0.001 and P=0.002, respectively), whereas CpG2006 did not (FIG. 1 A). Furthermore, LD of IC31® elicited higher Ab response than the HD of IC31® (P=0.003) or the CpG2006 (P<0.001). Already, 3 weeks after priming the Pnc1-TT+LD of IC31® formulation had elicited significantly higher Ab levels than the other vaccine formulations, and this high level of Abs sustained the next week (FIG. 1 B), when the mice were challenged with serotype 1 pneumococci.

The same pattern was seen for the IgG subclasses, that is LD of IC31 elicited higher IgG1 (P=0.003), IgG2a (P=0.038), IgG2b (P=0.001) and IgG3 (P=0.01) than the HD of IC31® (Table 1).

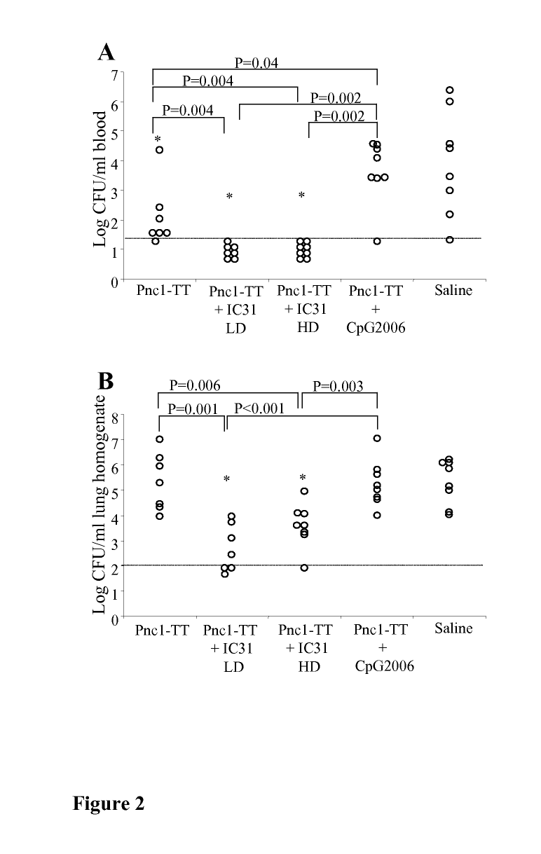

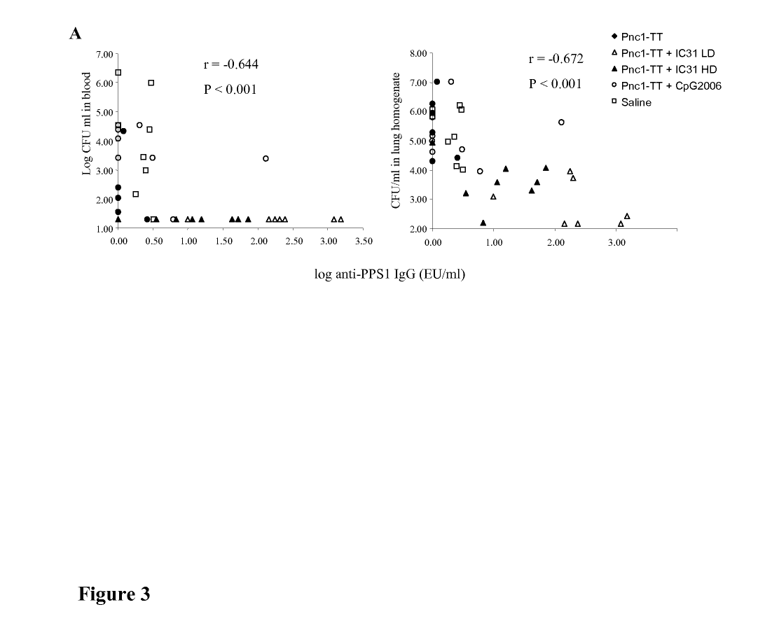

Four weeks after the immunization mice were challenged intranasally with S. pneumoniae of serotype 1 (3.6×10⁷ CFU/mouse). Twenty four hours later the mice were bled, sacrificed, the lungs removed and bacteremia and lung infection evaluated by counting CFU. Mice receiving Pnc1-TT mixed with either LD or HD of IC31® had no pneumococci in blood, in contrast to the groups receiving Pnc1-TT alone (P=0.004) or mixed with CpG2006 (P=0.002). The mice receiving Pnc1-TT alone had significantly reduced bacteremia (P=0.04) compared to the saline group whereas the group receiving Pnc1-TT+CpG2006 did not (FIG. 2A). Both doses of IC31® significantly reduced the lung infection compared to Pnc1-TT alone although only three out of seven mice in LD IC31® group and one out of eight mice in the HD IC31® had completely cleared the bacteria from the lungs. There was no reduction of lung infection in either the group receiving Pnc1-TT alone or mixed with CpG2006 (FIG. 2B). There was a highly significant negative correlation between Ab levels and CFU of pneumococci in lungs and blood (r=−0.672, p<0.001 and r=−0.644, p<0.001, respectively) (FIG. 3). In summary, even after one single neonatal immunization with Pnc1-TT and IC31® the mice were fully protected against bacteremia and had significantly reduced bacteria in lungs compared to mice receiving Pnc1-TT alone.

To evaluate the safety of IC31 in the neonatal mice they were weighted at the end of the experiment. No significant reduction in weight was observed in groups receiving IC31® or CpG2006 compared to the group receiving Pnc1-TT alone. However, all the immunized groups were slightly lighter than the saline group, which was significant for the groups receiving Pnc1-TT alone or with HD IC31® (Table 1).

### Example 2

### Two Doses of Pnc1-TT with Low Dose IC31v Elicited Rapid and Strong Ab Response with Full Protection Against Pneumococcal Lung Infection and Bacteremia

Mice were immunized twice, 7 days old and again 16 days later with Pnc1-TT with or without LD or HD IC31®. Because of higher Ab response elicited by LD than HD IC31® with Pnc1-TT given once at 7 days of age in the first set of experiments; one group received age dependent doses, i.e. LD at the first immunization and HD at the second. Saline was used as a negative control. As before, the mice were bled two weeks after the first immunization and weekly thereafter.

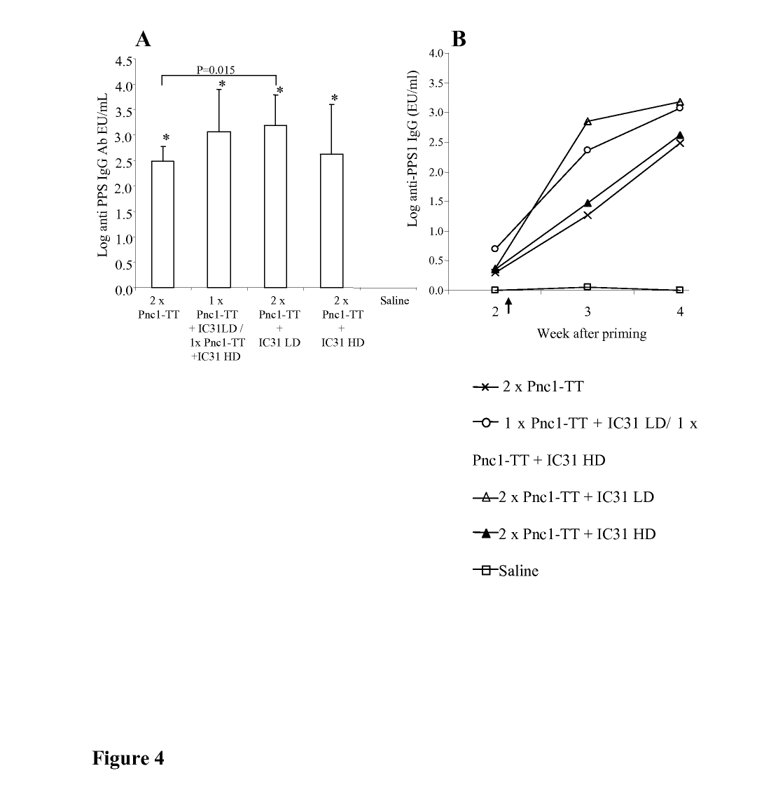

All the immunized groups had significantly higher Ab response than the saline group two weeks after the second immunization, but there was no difference in the Ab levels between the groups receiving adjuvants. The group receiving 2 doses of Pnc1-TT and LD IC31® was the only group eliciting significantly (P=0.015) higher Ab response than the group receiving 2 doses of Pnc1-TT alone (FIG. 4A). No difference was observed in final Ab levels in mice receiving 2 doses of Pnc1-TT with LD or HD IC31®. However, in this experiment, mice primed with LD IC31® and given either HD or LD IC31® for the 2nd immunization had more rapid response, reflected in significantly higher Ab levels 3 weeks after priming compared to mice receiving Pnc1-TT alone (P=0.021 and P=0.001, respectively), whereas those receiving 2 doses of HD IC31® did not (FIG. 4B). However, the more rapid Ab response induced by the first dose of LD compared to HD IC31® was not significant in another comparable experiment and in three identical experiments the Ab levels were comparable two weeks after the second immunization in the groups receiving HD or LD of IC31® (data not shown). This demonstrates that the LD of IC31 is sufficient and optimal to elicit maximum Ab response to Pnc1-TT in neonatal mice.

Following two immunizations all the groups had significantly higher IgG1 levels than the saline group, but compare to the Pnc1-TT group, only the group receiving 2 doses of LD IC31® had significantly higher IgG1 (P=0.01). IgG2a and IgG2b levels were low with high variation within each group. All groups receiving IC31® had significantly higher IgG2b (P<0.01) than the saline group and the group receiving 2×Pnc1-TT+HD IC31® had significantly higher IgG2b (P=0.003) than the Pnc1-TT group (Table 1). IgG3 levels tended to be higher in the immunized groups than the saline group but due to a high variation this was only significant for the group receiving 2×Pnc1-TT+LD IC31® (P=0.01).

The weight of mice receiving IC31 was not reduced compared to the groups receiving Pnc1-TT alone or saline (Table 1).

There was a significant negative correlation between Ab levels and CFU of pneumococci in lungs and blood of mice immunized twice with Pnc1-TT with or without adjuvants (r=−0697, p<0.001 and r=−0.697, p<0.001 respectively) (data not shown).

TABLE 1
PPS1 specific IgG subclasses and weight of mice 2 weeks after one or two immunizations

| 1^{st} | 2^{nd} | Mean Ab levels (EU/ml) | | | | Weight |
|---|---|---|---|---|---|---|
| immunization | immunization | IgG1 | IgG2a | IgG2b | IgG3 | (g), mean ± SD |
| Pnc1-TT | — | ND | ND | ND | ND | 23 ± 1 |
| Pnc1-TT + IC31 | — | 2.27 ± 0.82 | 0.38 ± 0.44 | 0.62 ± 0.28 | 1.49 ± 1.02 | 25 ± 1 |
| L.D | | | | | | |
| Pnc1-TT + IC31 | — | 1.14 ± 0.22 | ND | 0.00 ± 0.07 | ND | 24 ± 1 |
| H.D | | | | | | |
| Pnc1-TT + | — | 0.04 ± 0.11 | ND | ND | 0.15 ± 0.00 | — |
| CpG2006 | | | | | | |
| Saline | — | 0.01 ± 0.01 | 0.01 ± 0.01 | ND | ND | 26 ± 1 |
| Pnc1-TT | Pnc1-TT | 2.21 ± 0.29 | ND | 0.01 ± 0.02 | 0.64 ± 0.70 | 27 ± 2 |
| Pnc1-TT + IC31 | Pnc1-TT + IC31 | 2.63 ± 0.94 | 0.08 ± 0.19 | 0.43 ± 0.50 | 0.55 ± 0.80 | 28 ± 2 |
| L.D | H.D | | | | | |
| Pnc1-TT + IC31 | Pnc1-TT + IC31 | 2.94 ± 0.60 | 0.10 ± 0.28 | 0.31 ± 0.28 | 1.60 ± 1.10 | 26 ± 2 |
| L.D | L.D | | | | | |
| Pnc1-TT + IC31 | Pnc1-TT + IC31 | 2.33 ± 1.10 | 0.14 ± 0.26 | 0.52 ± 0.53 | 1.01 ± 1.20 | 26 ± 2 |
| H.D | H.D | | | | | |
| Saline | Saline | ND | ND | ND | ND | 24 ± 2 |

Mice were immunized once or twice as indicated.
IgG1-, IgG2a-, IgG2b- and IgG3- anit PPS1 were measured. Results are expressed as mean ± SD of log EU/mL.
The weight of mice (g) was measured to evaluate the safety of the vaccine combinations. Results are shown as mean ± SD of g.
ND means not detected.

1.-16. (canceled)

17. A composition comprising:
at least one polysaccharide-polypeptide conjugate;
at least one peptide comprising a sequence R₁—XZXZ_{N}XZX—R₂ (SEQ ID NOs: 1-5), whereby N is a whole number between 3 and 7, X is a positively charged natural and/or non-natural amino acid residue, each Z is independently an amino acid residue further defined as L, V, I, F and/or W, and R₁ and R₂ are independently —H, —NH₂, —COCH₃, —COH, a peptide with up to 20 amino acid residues or a peptide reactive group or a peptide linker with or without a peptide, X—R₂ is an amide, ester or thioester of the C-terminal amino acid residue of the peptide (“Peptide A”); and
at least one immunostimulatory oligodeoxynucleic acid molecule (ODN) having the structure of the formula (I) 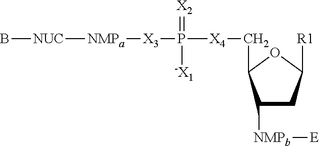
wherein:
R1 is hypoxanthine or uracile;
any X is O or S;
any NMP is a 2′ deoxynucleoside monophosphate or monothiophosphate, further defined as deoxyadenosine-, deoxyguanosine-, deoxyinosine-, deoxycytosine-, deoxyuridine-, deoxythymidine-, 2-methyl-deoxyinosine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine-monophosphate or -monothiophosphate;
NUC is a 2′ deoxynucleoside, further defined as deoxyadenosine-, deoxyguanosine-, deoxyinosine-, deoxycytosine-, deoxyinosine-, deoxythymidine-, 2-methyl-deoxyuridine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine;
a and b are integers from 0 to 100 with the proviso that a+b is between 4 and 150; and
B and E are common groups for 5′ or 3′ ends of nucleic acid molecules (“I-/U-ODN”).

18. The composition of claim 17, wherein N is 5.

19. The composition of claim 17, wherein Peptide A is KLKLLLLLKLK (SEQ ID NO: 6).

20. The composition of claim 17, wherein I-/U-ODN is Oligo(dIdC)₁₃.

21. The composition of claim 17, wherein the polysaccharide-polypeptide conjugate comprises a S. pneumoniae capsular polysaccharide.

22. The composition of claim 17, wherein the polysaccharide-polypeptide conjugate comprises a tetanus toxoid.

23. A pharmaceutical composition comprising the composition of claim 17.

24. The pharmaceutical composition of claim 23, further comprising a pharmaceutically acceptable carrier or excipient.

25. The pharmaceutical composition of claim 23, comprising the composition in an amount suitable for prevention of an infection with S. pneumoniae.

26. The pharmaceutical composition of claim 23, further defined as adapted for administration to a human toddler, infant, or neonate.

27. A method for the prevention of a bacterial infection in a subject, comprising administering a prophylactically effective amount of a composition of claim 17 to a subject in need thereof.

28. The method of claim 27, wherein the subject is a human subject.

29. The method of claim 28, wherein the subject is a human toddler, infant, or neonate.

30. The method of claim 27, wherein the bacterial infection is an infection with S. pneumoniae.

31. A method of improving protective efficacy of a composition comprising a polysaccharide-polypeptide conjugate comprising administering a combination of Peptide A and a I-/U-ODN, as defined in claim 17, to a subject in need thereof.

32. The method of claim 31, further comprising preserving or enhancing a type 2 response of said composition.
